(19) United States
(12) Patent Application Publication
(10) Pub. No.: US 2022/0273173 A1
(43) Pub. Date: Sep. 1, 2022

(54) NONINVASIVE DETECTION AND/OR TREATMENT OF MEDICAL CONDITIONS

(71) Applicant: Not Impossible, LLC, Venice, CA (US)

(72) Inventors: Antonio Rafael Rodriguez Chapa, Austin, TX (US); Daniel Biscaro Loureiro, Philadelphia, PA (US)

(21) Appl. No.: 17/652,867

(22) Filed: Feb. 28, 2022

Related U.S. Application Data
(60) Provisional application No. 63/200,289, filed on Feb. 26, 2021.

(51) Int. Cl. A61B 5/00; A61B 5/11

(52) U.S. Cl. CPC A61B 5/0051; A61B 5/0004; A61B 5/002; A61B 5/1101; A61B 5/7267; A61B 5/6831; A61B 5/6824; A61B 5/6829

(57) ABSTRACT

Noninvasive treatment (e.g., neuromodulation) can be achieved using vibrational energy applied via one or more wearable devices. A noninvasive treatment device includes a vibrational actuator disposed within a housing that can be secured to a user's body at or adjacent a treatment site. One or more sensors can collect physiological data before, during, or after application of vibrational energy to monitor a user's condition. Machine learning or other suitable approaches can be used to analyze sensor data to detect medical conditions and/or to effect treatment of medical conditions using devices as described herein.

### CROSS-REFERENCE TO RELATED APPLICATIONS

This application claims priority to U.S. Provisional Application No. 63/200,289, filed Feb. 26, 2021, which is hereby incorporated by reference in its entirety.

### TECHNICAL FIELD

The present technology relates to noninvasive detection and/or treatment devices and associated systems and methods of use. In particular, the present technology is directed to devices that use wearable sensors to detect and treat conditions, for example using vibrational energy to noninvasively modulate nerve activity.

### BACKGROUND

Implantable neuromodulation devices have been developed to treat pain, movement disorders, functional disorders, spasticity, cancer, cardiac disorders, and various other medical conditions. Implantable neuromodulation systems generally have an implantable signal generator and one or more leads that deliver electrical pulses to neurological tissue or muscle tissue. Use of these devices requires invasive surgical procedures that risk damage to surrounding tissue and involve extended recovery periods. Noninvasive neuromodulation systems avoid many of the problems associated with surgical procedures for implantable devices, but tend to be less effective in treating movement disorders or other conditions. Accordingly, there is a need for improved systems and methods for noninvasive treatment such as noninvasive neuromodulation.

### SUMMARY

The present technology is directed to noninvasive treatment devices, systems, and methods. The subject technology is illustrated, for example, according to various aspects described below, including with reference to FIGS. 1-6. Various examples of aspects of the subject technology are described as numbered clauses (1, 2, 3, etc.) for convenience. These are provided as examples and do not limit the subject technology. These clauses can be combined in any order and in any combination.

Clause 1. A noninvasive treatment device, comprising: a vibration actuator configured to deliver vibrational energy to a treatment site of a user; a sensor configured to obtain physiological data from the user; and a controller communicatively coupled to the sensor, the controller configured to: receive the physiological data from the sensor; analyze the physiological data; and based on the analysis, modulate delivery of vibrational energy via the vibration actuator.

Clause 2. The device of any one of the Clauses herein, wherein analyzing the physiological data comprises applying one or more artificial intelligence (AI) or machine-learning (ML) algorithms.

Clause 3. The device of any one of the Clauses herein, wherein modulating delivery comprises initiating delivery of vibrational energy via the vibration actuator.

Clause 4. The device of any one of the Clauses herein, wherein modulating delivery comprises ceasing delivery of vibrational energy via the vibration actuator.

Clause 5. The device of any one of the Clauses herein, wherein modulating delivery comprises varying an intensity of vibrational energy delivered via the vibration actuator.

Clause 6. The device of any one of the Clauses herein, wherein modulating delivery comprises varying a frequency of vibrational energy delivered via the vibration actuator.

Clause 7. The device of any one of the Clauses herein, wherein the sensor comprises an accelerometer.

Clause 8. The device of any one of the Clauses herein, wherein the sensor comprises a temperature sensor.

Clause 9. The device of any one of the Clauses herein, wherein the sensor comprises a blood pressure sensor.

Clause 10. The device of any one of the Clauses herein, wherein the physiological data comprises movement data.

Clause 11. The device of any one of the Clauses herein, wherein the physiological data comprises an indication of a user tremor.

Clause 12. The device of any one of the Clauses herein, wherein the physiological data comprises an indication of a user fall.

Clause 13. The device of any one of the Clauses herein, further comprising a fastener configured to secure the device against a user's body at or adjacent the treatment site;

Clause 14. The device of any one of the Clauses herein, wherein the fastener comprises a band configured to removably secure the device against the user's body at or adjacent the treatment site.

Clause 15. The device of any one of the Clauses herein, wherein the treatment device comprises a housing enclosing the vibration actuator, the sensor, and the controller, and wherein the housing is removably coupleable to the fastener.

Clause 16. The device of any one of the Clauses herein, wherein the sensor and the controller are enclosed within a common housing.

Clause 17. A treatment system comprising a plurality of the treatment devices of any one of the Clauses herein, each of the devices configured to be disposed over a different treatment site of the user.

Clause 18. A method for treatment (e.g., modulating nerve activity of) a user, the method comprising: disposing a treatment device adjacent a treatment site of the user; applying vibrational energy to the treatment site via the treatment device; sensing physiological data of the user via the treatment device; analyzing the physiological data; based on the analysis, modulating the vibrational energy applied to the treatment site.

Clause 19. The method of any one of the Clauses herein, wherein modulating the vibrational energy applied comprises initiating application of the vibrational energy.

Clause 20. The method of any one of the Clauses herein, wherein modulating the vibrational energy applied comprises ceasing application of the vibrational energy.

Clause 21. The method of any one of the Clauses herein, wherein modulating the vibrational energy applied varying an intensity of vibrational energy applied.

Clause 22. The method of any one of the Clauses herein, wherein modulating the vibrational energy applied comprises varying a frequency of vibrational energy applied.

Clause 23. The method of any one of the Clauses herein, wherein the sensing the physiological data comprises using an accelerometer.

Clause 24. The method of any one of the Clauses herein, wherein the sensing the physiological data comprises using a temperature sensor.

Clause 25. The method of any one of the Clauses herein, wherein the sensing the physiological data comprises using a blood pressure sensor.

Clause 26. The method of any one of the Clauses herein, wherein the physiological data comprises movement data.

Clause 27. The method of any one of the Clauses herein, wherein the physiological data comprises an indication of a user tremor.

Clause 28. The method of any one of the Clauses herein, wherein the physiological data comprises an indication of a user fall.

Clause 29. The method of any one of the Clauses herein, further comprising securing the treatment device against the user's body at or adjacent the treatment site via a fastener.

Clause 30. The method of any one of the Clauses herein, wherein the fastener comprises a band configured to removably secure the device against the user's body at or adjacent the treatment site.

Clause 31. The method of any one of the Clauses herein, wherein the treatment device comprises a housing enclosing the vibration actuator, the sensor, and the controller, and wherein the housing is removably coupleable to the fastener.

Clause 32. The method of any one of the Clauses herein, wherein the sensor and the controller are enclosed within a common housing.

Clause 33. The method of any one of the Clauses herein, further securing the treatment device against the user's body at or adjacent the treatment site.

Clause 34. The method of any one of the Clauses herein, further comprising: disposing a plurality of treatment devices adjacent different treatment sites of the user; and applying vibrational energy to each of the treatment site via the corresponding treatment devices.

Clause 35. A noninvasive treatment system, comprising a plurality of treatment devices each configured to be disposed over a respective treatment site of a user, wherein each of the treatment devices comprises: a vibration actuator configured to deliver vibrational energy to the respective treatment site of the user; one or more sensors configured to obtain physiological data from the user, the physiological data including at least movement data; and a controller communicatively coupled to the one or more sensors, the controller configured to: receive the physiological data from the one or more sensors; analyze the physiological data to determine that a tremor condition has been detected; based on the determination that the tremor is occurring, initiate delivery of vibrational energy via the vibration actuator; while delivering vibrational energy via the vibration actuator, receive additional physiological data from the sensor; analyze the additional physiological data to determine that the tremor has ceased or decreased in severity; and based on the determination that the tremor condition is no longer detected, ceasing the delivery of vibrational energy via the vibration actuator.

Clause 36. The noninvasive treatment system of any one of the Clauses herein, wherein analyzing the physiological data to determine that a tremor is occurring comprises using a decision tree to evaluate the physiological data.

Clause 37. The noninvasive treatment system of any one of the Clauses herein, wherein each of the treatment devices further comprises an input mechanism, and wherein the controller is further configured to: while delivering vibrational energy via the vibration actuator, receiving a user input via the input mechanism; and responsive to the user input, ceasing the delivery of vibrational energy via the vibration actuator.

Clause 38. The noninvasive treatment system of any one of the Clauses herein, wherein the input mechanism comprises a touch-sensitive element, and wherein the user input comprises a user tapping the touch-sensitive element.

Clause 39. The noninvasive treatment system of any one of the Clauses herein, wherein the controller is an ultra-low power controller configured to analyze the physiological data to determine that the tremor is occurring using less than about 1 milliwatt of power.

Clause 40. The noninvasive treatment system of any one of the Clauses herein, wherein the controller is an ultra-low power controller configured to analyze the physiological data to determine that the tremor is occurring using less than about 1 microwatt of power.

Clause 41. The noninvasive treatment system of any one of the Clauses herein, wherein the one or more sensors comprises an accelerometer and a gyroscope, and wherein the physiological data comprises accelerometer motion data along three axes and gyroscope rotation data along three axes.

Clause 42. The noninvasive treatment system of any one of the Clauses herein, wherein at least some of the treatment devices comprise a housing coupled to a fastener configured to secure the housing against the user's wrists or ankles.

Clause 43. The noninvasive treatment system of any one of the Clauses herein, wherein the housing encloses the vibration actuator, the one or more sensors, and the controller.

Clause 44. The noninvasive treatment system of any one of the Clauses herein, wherein the treatment devices comprises at least four treatment devices configured to be disposed over a user's wrists and ankles, respectively, and wherein each of the treatment devices collects and analyzes physiological data independently of the other treatment devices.

Clause 45. A treatment device, comprising: a vibration actuator configured to deliver vibrational energy to a treatment site of a user; one or more sensors configured to obtain physiological data from the user; and a controller communicatively coupled to the one or more sensors, the controller configured to: receive the physiological data from the one or more sensors; analyze the physiological data; and based on the analysis, modulate delivery of vibrational energy via the vibration actuator.

Clause 46. The treatment device of any one of the Clauses herein, wherein analyzing the physiological data comprises applying a classification algorithm to the physiological data to make a tremor determination.

Clause 47. The treatment device of any one of the Clauses herein, wherein modulating delivery comprises initiating delivery of vibrational energy via the vibration actuator.

Clause 48. The treatment device of any one of the Clauses herein, wherein modulating delivery comprises at least one of: initiating delivery of vibrational energy via the vibration actuator, ceasing delivery of vibrational energy via the vibration actuator, varying an intensity of vibrational energy delivered via the vibration actuator, or varying a frequency of vibrational energy delivered via the vibration actuator.

Clause 49. The device of any one of the Clauses herein, wherein the one or more sensors comprises at least one of: an accelerometer, a gyroscope, a temperature sensor, or a blood pressure sensor.

Clause 50. The device of any one of the Clauses herein, wherein the physiological data comprises an indication of a user tremor.

Clause 51. A method for treatment of a tremor, the method comprising: disposing a wearable treatment device adjacent a treatment site of the user, the treatment device comprising a vibration actuator, one or more sensors, and a controller; sensing physiological data via the one or more sensors of the treatment device, the physiological data including at least movement data; analyzing, via the controller, the physiological data to make a determination that a tremor condition has been detected; after making the determination, applying vibrational energy to the treatment site via the vibration actuator of the treatment device; sensing additional physiological data of the user via the one or more sensors of the treatment device; analyzing, via the controller, the additional physiological data to make a determination that the tremor condition is no longer detected; and after making the determination that the tremor condition is no longer detected, ceasing applying vibrational energy to the treatment site via the vibration actuator of the treatment device.

Clause 52. The method of any one of the Clauses herein, wherein the treatment device further comprises an input mechanism, and wherein the method further comprises: while applying vibrational energy via the vibration actuator, receive a user input via the input mechanism; and responsive to the user input, ceasing applying vibrational energy via the vibration actuator.

Clause 53. The method of any one of the Clauses herein, wherein the controller consumes less than about 1 milliwatt of power in analyzing the physiological data.

Clause 54. The method of any one of the preceding Clauses, wherein the one or more sensors comprises an accelerometer and a gyroscope, wherein the physiological data comprises accelerometer motion data along three axes and gyroscope rotation data along three axes, and wherein analyzing the physiological data comprises applying a decision tree to the accelerometer motion data and the gyroscope rotation data.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a schematic diagram of a system configured in accordance with embodiments of the present technology.

FIG. 1B is a schematic diagram of a user utilizing the system shown in FIG. 1A. FIG. 2 is a perspective view of an example device in accordance with embodiments of the present technology.

FIGS. 3A and 3B are perspective top and bottom views, respectively, of an example charging base for devices in accordance with embodiments of the present technology.

FIG. 4 illustrates a device being removably mated with a charging base in accordance with embodiments of the present technology.

FIG. 5 is a flow diagram of a method for noninvasively modulating one or more nerves at a treatment site of a user, in accordance with embodiments of the present technology.

FIG. 6 illustrates example features and corresponding data for classifying tremor and non-tremor conditions based on sensor data in accordance with embodiments of the present technology.

### DETAILED DESCRIPTION

### I. Overview

Neuromodulation (e.g., stimulation or inhibition of nerves) can be used to treat a variety of conditions, including chronic pain, movement disorders, functional disorders, spasticity, mental disorders, cardiac disorders, and various other medical conditions. Mechanical modulation of nerves can be achieved using vibrational actuators coupled to the user's body at appropriate treatment sites. For example, a wearable neuromodulation device can include a vibrational actuator configured to be fastened against the user's body using straps or by being embedded in garments (e.g., gloves, socks, vests, headbands). In some instances, a user may wear a plurality of neuromodulation devices at different treatment locations (e.g., on both wrists), and such devices may work in concert to provide the desired therapeutic effect.

Although many of the embodiments are described herein with respect to systems, devices, and methods for noninvasive neuromodulation, the technology is applicable to other applications and/or other approaches, such as noninvasive application of vibrational energy for other non-neuromodulation applications, such as to augment musical listening experiences, as interactive multi-media, or any other application. Additionally, systems, devices, and methods of the present technology can be applied for therapeutic effect that does not involve (or does not primarily rely on) neuromodulation. For example, in some embodiments the vibrational actuators may instantiate one or more therapeutic effects independent of modulating nerve activity.

Vibrational energy can be applied in a number of ways, for example varying the frequency, intensity, duty cycle, duration, or other treatment parameters. In some embodiments, the neuromodulation device(s) can include one or more sensors configured to detect certain physiological parameters. For example, the sensors can include accelerometers (or other motion sensors), altimeters, thermometers, blood pressure monitors, cardiac sensors (e.g., ECG sensors), pulse oximeters, blood glucose sensors, or any other suitable sensors that can detect physiological parameters indicative of a user's health or physical condition. In various examples, the sensor(s) can be configured to detect a user's movement, body temperature, cardiac rhythm, blood oxygen levels, blood sugar levels, or any other suitable physiological parameter. The neuromodulation provided by the wearable device(s) can be modified based on feedback from the one or more sensors. For example, neuromodulation can be initiated (e.g., the device can begin to apply vibrational energy) or ceased in response to certain physiological conditions being met. As another example, the frequency, intensity, duty cycle, or other waveform parameters may be varied in response to detected physiological parameters. In some embodiments, the physiological parameters can be collected across a wide range of users and used to train a machine-learning classification algorithm (e.g., a neural network model) that can be used to determine an appropriate neuromodulation to be applied given a particular parameter or set of parameters detected via the sensor(s). In various embodiments, machine learning techniques (e.g., the decision tree model described with respect to the Appendix) can be used to evaluate sensor data in real-time or nearly real-time to identify medical conditions (e.g., a tremor) and optionally to initiate the appropriate treatment.

### II. Example Noninvasive Treatment Systems and Devices

### Treatment System Overview

The following discussion provides a brief, general description of a suitable environment in which the present technology may be implemented. Although not required, aspects of the technology are described in the general context of computer-executable instructions, such as routines executed by a general-purpose computer. Aspects of the technology can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, a short-range radio network (e.g., via Bluetooth)). In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In some embodiments, aspects of the technology may be distributed over the Internet or over other networks (e.g. a Bluetooth network) on a propagated signal on a propagation medium (e.g., an electromagnetic wave(s), a sound wave) over a period of time, or may be provided on any analog or digital network (packet switched, circuit switched, or other scheme).

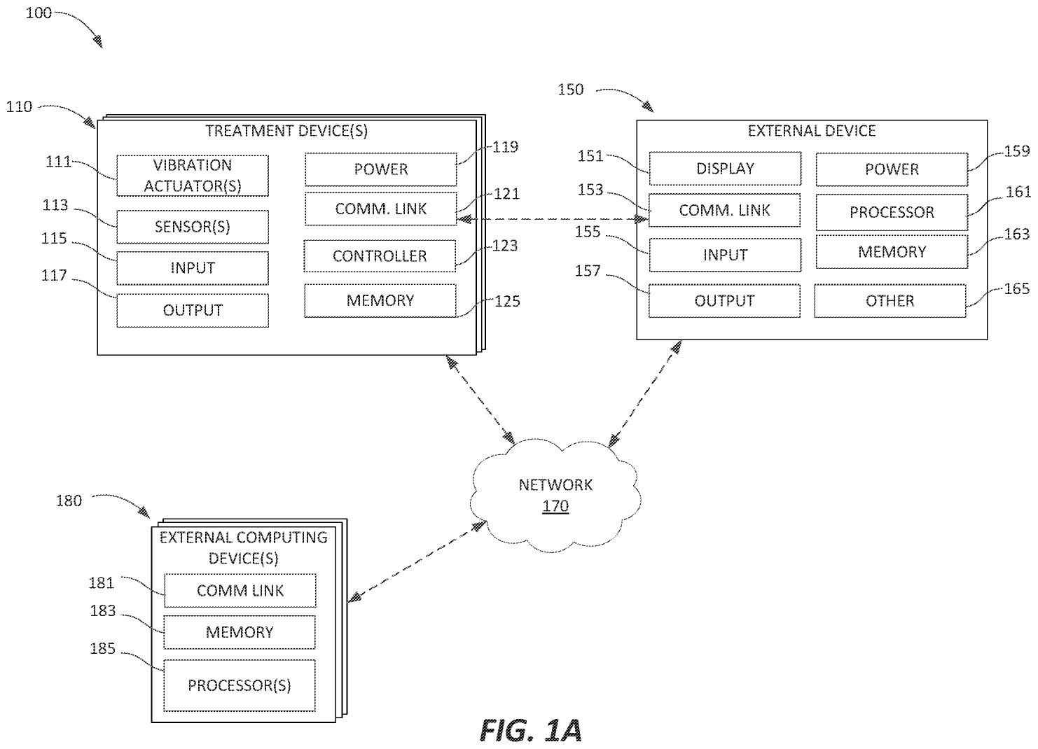

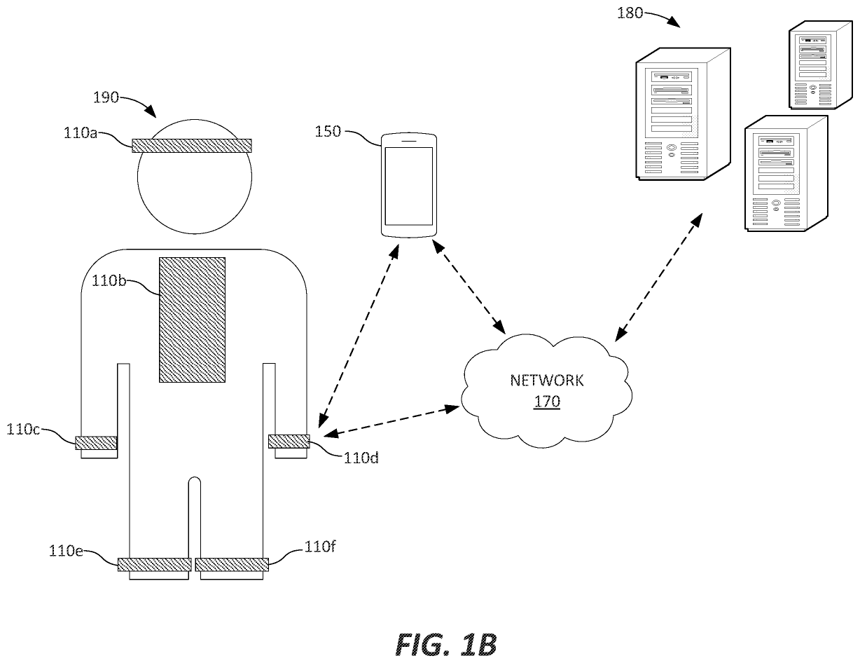

FIG. 1A is a schematic diagram of treatment system 100 configured in accordance with an embodiment of the disclosed technology. Although the system 100 is shown with certain devices for purposes of explanation, in various examples any one or more of the devices shown in FIG. 1A can be omitted. Similarly, although the devices shown in FIG. 1A are illustrated as including certain components, in various examples any one or more of the particular components within these devices can be omitted (e.g., the treatment device 110 may omit the sensor(s) 113). Moreover, any of the devices can include additional components not specifically shown here.

In the illustrated embodiment, the treatment device 110 comprises one or more vibration actuator(s) 111, one or more sensor(s) 113, input 115, output 115, a power source 119, a communications link 121, a controller 123, and a memory 125. The treatment device 110 is configured to be coupled to a user for energy delivery to modulate nerve activity at a treatment site. For example, the treatment device 110 can may be removably worn by the user, for example positioned directly over the user's ankle or wrist and held in place via a band or other fastener.

The vibration actuator(s) 111 can be any suitable component or combination of components configured to supply vibrational energy that can be applied to a treatment site. For example, in various examples, the vibration actuator(s) 111 can include a linear resonant actuator (LRA), an eccentric rotating mass (ERM) actuator, a piezoelectric actuator, a speaker, or any other suitable actuator capable of delivering vibrational energy. In some embodiments, the actuators can be configured to supply vibrational energy over any suitable range of frequencies, for example between about 30 Hz to about 1000 Hz, or in some cases between about 30 to about 250 Hz. In some embodiments, the vibrational actuators can be configured to vibrate primarily within an x-y plane, with little or no vibration or oscillation along the z-axis. In use, the treatment device 110 can be placed against the user's skin with the x-y plane being substantially parallel to the user's skin (i.e., the z-axis is substantially perpendicular to the user's skin). By orienting the vibration actuator 111 such that the primary mode of vibration is oriented parallel to the user's skin, patient comfort can be improved (e.g., by avoiding the pulsing or throbbing sensation of a high intensity z-axis vibration). In at least some embodiments, the vibration actuator 111 can be configured such that the frequency of vibration and the intensity of vibration is effectively decoupled (as opposed to, for example, some ERM actuators in which intensity is increased in direct proportion with frequency). This can permit the intensity of energy applied to the treatment site and the frequency of vibrational energy to be varied independently of one another to achieve the desired clinical benefit.

In various embodiments, several levels of intensity can be obtained by the amount of voltage supplied to the actuators. Different patterns can be provided achieving variance in rhythm and pulses on the intensity and/or frequency of the vibrations. Time of exposure can be controlled through a provided timer. All of the four actuators can be fired at once with the same or independent patterns/frequencies/intensities according to the desired treatment. Cycles can be set to keep devices operating for a period of time letting the user rest to test out different wash-out times. In some embodiments, a random frequency mode can be set to refresh the receptors on the skin. Additionally or alternatively, a ramp mode can be set so frequency goes up in adjustable slow increments so the researcher can determine what frequency works best for each patient without having to increase each value manually.

The sensor(s) 113 can include a number of different sensors and/or types of sensors. For example, the sensor(s) 113 can include a plurality of electrodes, an accelerometer, a blood pressure sensor, a pulse oximeter, an ECG sensor or other heart-recording device, an EMG sensor or other muscle-activity recording device, a temperature sensor, a skin galvanometer, hygrometer, altimeter, gyroscope, magnetometer, proximity sensor, hall effect sensors, or any other suitable sensor for monitoring physiological characteristics of the user. These particular sensors are exemplary, and in various embodiments the sensors employed can vary. In some embodiments, the treatment device 110 omits the sensors 113 altogether.

In some embodiments, the power source 119 can be rechargeable, for example using inductive charging or other wireless charging techniques. Such rechargeability can facilitate long-term placement of the treatment device 110 on or within a user. The input 115 and output 117 components can include, for example one, or more buttons, keys, lights, microphones, speakers, ports (e.g., USB-C connector ports), touch-sensitive screen or other surface, etc.

In various embodiments, the memory 125 can take the form of one or more computer readable storage modules configured to store information (e.g., signal data, subject information or profiles, environmental data, treatment regimes, data collected from one or more sensing components, media files) and/or executable instructions that can be executed by the controller 123. The memory 125 can include, for example, instructions for causing the vibration actuators 111 to be activated, analyzing sensor data to evaluate the user's health status, etc. In some embodiments, the memory 125 stores data (e.g., sensor data acquired from the sensor(s) 113) used in the feedback techniques disclosed herein.

The communications link 121 enables the treatment device 110 to transmit to and/or receive data from external devices (e.g., external device 150 or external computing devices 180). The communications link 121 can include a wired communication link and/or a wireless communication link (e.g., Bluetooth, Near-Field Communications, LTE, 5G, Wi-Fi, infrared and/or another wireless radio transmission network).

The controller 123 can include, for example, a suitable processor or central processing unit (“CPU”) that controls operation of the treatment device 110 in accordance with computer-readable instructions stored on the memory 125. The controller 123 may be any logic processing unit, such as one or more CPUs, digital signal processors (DSPs), application-specific integrated circuits (ASICs), etc. The controller 123 may be a single processing unit or multiple processing units in a device or distributed across multiple devices. The controller 123 is connected to the memory 125 and may be coupled to other hardware devices, for example, with the use of a bus (e.g., a PCI Express or Serial ATA bus). The memory 125 can include read-only memory (ROM) and random access memory (RAM) or other storage devices, such as disk drives or SSDs, that store the executable applications, test software, databases and other software required to, for example, implement the various routines described herein, control device components, communicate and exchange data and information with remote computers and other devices, etc.

The controller 123 also includes drive circuitry configured to control operation of the vibration actuator(s) 111 of the treatment device 110. For example, the drive circuitry can be configured to deliver waveforms having predetermined and controllable parameters to one or more of vibration actuator(s) 111. The controller 123 can also be configured to initiate data collection via one or more sensor(s) 113. For example, the sensor(s) 113 of the treatment device 110 can detect physiological data of a user (e.g., motion data, temperature data, heart rhythm data, etc.). In some embodiments, this physiological data can be used in a feedback loop to affect operation of the vibration actuator(s) 111.

The treatment device 110 can be communicatively coupled to an external device 150, for example via a wireless connection. In some embodiments, the external device 150 can be a mobile device (e.g., a smartphone, tablet, smartwatch, etc.) or other computing device with which the user can interact. In operation, the treatment device 110 may receive input from and/or can be controlled by instructions from the external device 150. For example, the external device 150 can cause the treatment device 110 to initiate or cease energy delivery, can schedule energy delivery sessions, can vary parameters of the energy delivery (e.g., intensity, frequency, etc.), and/or provide other control instructions to the treatment device 110. Additionally or alternatively, the external device 150 may output user prompts which can be synchronized with data collection via the treatment device 110. For example, the external device 150 may instruct the user to lift an arm, make a facial expression, etc., and the treatment device 110 may record physiological data (e.g., via sensor(s) 113) while the user performs the requested actions. Moreover, the external device 150 may itself analyze the user (e.g., the user's activity or condition in response to such prompts), for example using a camera to detect muscle tremors, using a microphone to detect slurred speech, or to detect any other indicia of health conditions. In some embodiments, such indicia can be compared against baseline inputs (e.g., a stored baseline facial image or voice print with baseline speech recording).

The treatment device 110 and/or the external device 150 can also be communicatively coupled with one or more external computing devices 180 (e.g., over network 170). In some examples, the external computing devices 180 can take the form of servers, personal computers, tablet computers or other computing devices associated with one or more healthcare providers (e.g., hospitals, medical data analytics companies, device manufacturers, etc.). These external computing devices 180 can collect data recorded by the treatment device 110 and/or the external device 150. In some embodiments, such data can be anonymized and aggregated to perform large-scale analysis (e.g., using machine-learning techniques or other suitable data analysis techniques) to develop and improve treatment algorithms using data collected by a large number of treatment devices 110. Additionally, the external computing devices 180 may transmit data to the external device 150 and/or the treatment device 110. For example, an updated algorithm for treating one or more health conditions may be developed by the external computing devices 180 (e.g., using machine learning or other techniques) and then provided to the treatment device 110 and/or the external device 150 via the network (e.g., as an over-the-air update), and installed on the treatment device 110 and/or external device 150.

In some embodiments, the system 100 can also include additional devices, for example implantable neuromodulation devices, cardiac monitors, an implantable pacemaker, an implantable cardiac defibrillator, a cardiac resynchronization therapy (CRT) device (e.g., CRT-D defibrillator or CRT-P pacemaker), a neurostimulator, a deep-brain stimulation device, a nerve stimulator, a drug pump (e.g., an insulin pump), a glucose monitor, a fitness monitor, a nutrition device, etc.

The treatment device 110 may be configured to calculate physiological characteristics relating to one or more signals received from the sensor(s) 113. For example, the treatment device 110 may be configured to algorithmically determine the presence or absence of a muscle tremor, fall, or other health condition from the signal. In certain embodiments, the treatment device 110 initiate delivery of vibrational energy via the actuators 111 in response to sensor data (e.g., upon detecting muscle tremors using the accelerometer). In some embodiments, the sensing performed via the sensor(s) 113 can be modified in response to event detection, for example with an increased sampling rate or other modification.

As noted above, in some embodiments, the treatment device 110 may also communicate with an external device 150. The external device 150 can be, for example, a smartwatch, smartphone, laptop, tablet, desktop PC, or any other suitable computing device and can include one or more features, applications and/or other elements commonly found in such devices. For example, the external device 150 can include display 151, a communications link 153 (e.g., a wireless transceiver that may include one or more antennas for wirelessly communicating with, for example, other devices, websites, and the treatment device 110). Communication between the external device 150 and other devices can be performed via, e.g., a network 170 (which can include the Internet, public and private intranet, a local or extended Wi-Fi network, cell towers, the plain old telephone system (POTS), etc.), direct wireless communication, etc. The external device 150 can additionally include well-known input components 131 and output components 133, including, for example, a touch screen, a keypad, speakers, a camera, etc.

In operation, the user may receive output or instructions from the external device 150 that are based at least in part on data received at the external device 150 from the treatment device 110. For example, the treatment device 110 may generate a fall alert based on analysis of data collected via sensor(s) 113. The treatment device 110 may then instruct the external device 150 to output an alert to the user (e.g., via display 151 and/or output 157) or another entity. In some embodiments, the alert can both be displayed to the user (e.g., via display 151 of the external device) and can also be transmitted to an appropriate emergency medical response service (e.g., a 9-1-1 call may be placed with location data from the external device 150 used to direct responders to locate the user), and/or to other healthcare provider entities or individuals (e.g. a hospital, emergency room, or physician). In some embodiments, embedded circuitry that provides location data (e.g., a GPS unit) can be included within the treatment device 110.

Additionally or alternatively, the external device 150 may output user prompts which may be used in conjunction with physiological data collection via the treatment device 110. For example, the external device 150 may instruct the user to perform an action (e.g., lift an arm, make a facial expression, etc.), and the external device 150 may record physiological data while the user performs the requested actions. In some embodiments, the external device 150 may itself analyze physiological parameters of the user, for example using a camera to detect muscle tremor or other indicia of health conditions. In some embodiments, such physiological data collected via the external device 150 can be combined with data collected via the sensor(s) 113 and analyzed together to make a determination of a user's health status.

As noted previously, the external computing device(s) 180 can take the form of servers or other computing devices associated with healthcare providers or other entities. The external devices can include a communications link 181 (e.g., components to facilitate wired or wireless communication with other devices either directly or via the network 170), a memory 183, and processing circuitry 185. These external computing devices 180 can collect data recorded by the treatment device 110 and/or the external device 150. In some embodiments, such data can be anonymized and aggregated to perform large-scale analysis (e.g., using machine-learning techniques or other suitable data analysis techniques) to develop and improve treatment algorithms using data collected by a large number of treatment devices 110 associated with a large population of users. Additionally, the external computing devices 180 may transmit data to the external device 150 and/or the treatment device 110. For example, an updated algorithm for treating conditions may be developed by the external computing devices 180 (e.g., using machine learning or other techniques) and then provided to the treatment device 110 and/or the external device 150 via the network 170, and installed on the recipient treatment device 110/150.

### Example Device Arrangements

FIG. 1B is a schematic view of the system 100 of FIG. 1A in conjunction with a user 190. As illustrated, the user 190 is outfitted with a plurality of treatment devices 110a-110f disposed at different locations about the user's body. In operation, some or all of the devices 110 can be in communication with external device 150 (e.g., either directly via Bluetooth or similar connection, or indirectly via network 170). As described previously, the external device 150 may also be in communication with external computing devices 180 (e.g., via network 170).

The treatment devices 110a-f illustrated in FIG. 1B represent a sample of potential placements and device configurations. As shown, the devices 110 include a first treatment device 110a in the form of a headband or other such configuration that is capable of being placed on or around the user's head. A second treatment device 110b is shown schematically in the form of a vest, shirt, or other garment-like device configured to be secured against a user's chest and/or back and to apply vibrational energy thereto. The third and fourth devices 110c and 110d take the form of wristbands, gloves, or other such configurations that facilitate securing the devices 110c and 110d against the user's wrists (or hands, forearms, etc.) to deliver vibrational energy thereto. Finally, the fifth and sixth devices 110e and 110f take the form of ankle bands, socks, or other such configurations that facilitate securing the devices 110e and 110f against the user's ankles (or feet, shins, etc.) to deliver vibrational energy thereto. Although these particular locations are illustrated here by way of example, various other body locations and/or garment/fastener types can be used. For example, one or more treatment devices 110 can be positioned anywhere along a user's arms, legs, back, spine, neck, hands, feet, groin, hips, thighs, face, head, etc. Additionally, the treatment devices 110 can be secured in location using straps, adhesives, by being secured into wearable garments (e.g., shirt, glove, sock, vest, hat, helmet, etc.) or using any other suitable temporary attachment technique.

FIG. 2 is a perspective view of a treatment device 110. The treatment device 110 shown in FIG. 2 includes a housing 201 that is removably coupled to a fastener 203. The housing 201 can contain the various components described above (e.g., actuator(s) 111, sensor(s) 113, power 119, communications link 121, controller 123, and memory 125). In some embodiments, the housing 201 also carries one or more inputs 115 such as buttons, dials, or microphones, and one or more outputs 117 such as lights or speakers. In some embodiments, the input elements can be used to turn the device on or off, set treatment times (e.g., activate the device for a predetermined time period, after which the device will automatically turn off), adjust the frequency, amplitude, waveform, and/or other parameters controlling the vibration transducer(s) 111. vThe housing 201 can be generally rectangular, for example having a height or thickness of approximately 1-5 cm, a width of approximately 5-10 cm, and a length of approximately 5-10 cm. However, other dimensions are possible in various embodiments. The housing 201 and the treatment device 110 can assume other shapes, configurations, and dimensions (both smaller and larger) depending on the particular components included, the desired treatment site, power requirements, and other parameters.

The fastener 203 can take the form of a band, for example that forms a circumferentially closed loop to facilitate securement around a user's wrist, ankle, or other body part. In the illustrated example, a single housing 201 is coupled to a single fastener 203. However, in other embodiments a single fastener 203 can carry two or more housings 201, each of which may include one or more vibration actuators 111 therein. For example, a single strap-like fastener 203 can carry two vibration actuators 111. Conversely, a single housing 201 can be secured via two or more fasteners 203. For example, a housing 201 can be secured to two fasteners 203 in the form of non-parallel loops, thereby facilitating placement at knees, shoulders, or other body parts that may be more difficult to secure with a single strap fastener 203. The fastener 203 can be secured in position using any suitable technique, for example hook-and-loop fasteners (e.g., VELCRO), buckles, clasps, adhesive, magnets, or any other suitable temporary attachment technique.

In operation, the treatment device 110 can be placed over a user's skin adjacent to a treatment site such that the housing 201 in contact with the user's skin. For example, the fastener 203 can be wrapped around a user's ankle, with the housing 201 in position against the user's skin at the ankle. Once in this position, vibrational energy provided by the actuator(s) 111 can modulate nerve activity at or adjacent the ankle.

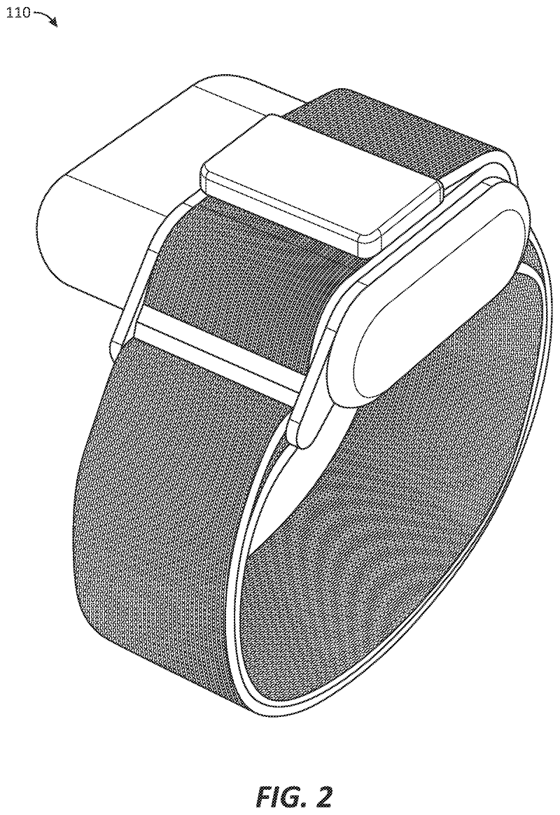

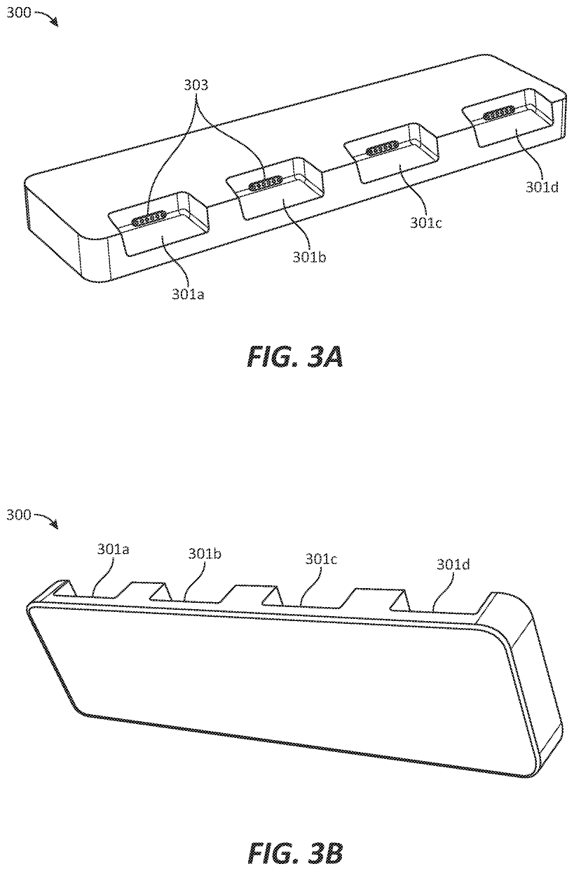

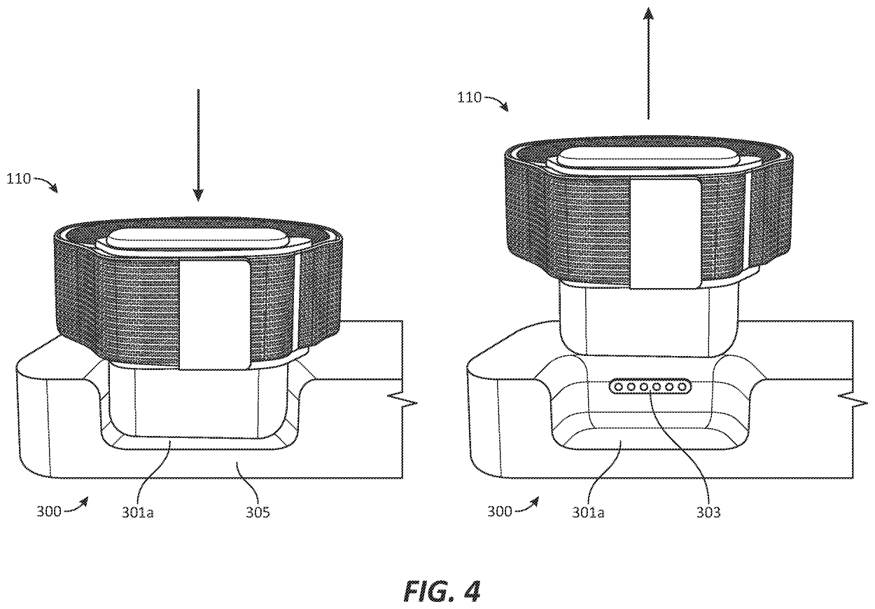

FIGS. 3A and 3B illustrate top and bottom perspective views, respectively, of a charging base 300 having a plurality of cradles 301a-d configured to removably receive a plurality of treatment devices 110 therein. FIG. 4 illustrates a single treatment device 110 being removably received within a charging cradle 301a of the base 300. Referring to FIGS. 3A-4 together, the base 300 can include an elongated body defining four cradles 301a-d in which the treatment devices 110 can be removably received. The cradles 301a-d can each include exposed electrical contacts 303 configured to be placed into direct physical contact with corresponding charging contacts of the treatment device 110 when placed within the cradle 301. The charging base 300 can have its own power source, which can take the form of a larger battery or a connection to facility power. As seen in FIG. 4, the base 300 can include an indicator light 305 that is illuminated when the corresponding treatment device 110 is charging within the cradle 301a.

### III. Example Methods of Use

The devices and methods disclosed herein may be applied to treatment of a wide variety of medical conditions. Without wishing to be bound by theory, it is believed that vibrational energy applied to certain treatment sites can non-invasively stimulate or inhibit nerves and provide effective treatment for a variety of different conditions.

### Example Treatment Conditions

Some of the conditions that may benefit from the application of vibrational energy to treatment site include movement disorders (e.g., involuntary tremors, dyskinesia, dystonia, freezing of gait, balance issues), mental and neurological disorders (e.g., cognitive load, Alzheimer' s, autism, post-traumatic stress disorder (PTSD)), high blood pressure, tinnitus, and spinal cord injuries. Additionally, there may be other conditions that benefit from the systems, devices, and methods disclosed herein, and particular conditions disclosed herein are intended to be exemplary only.

Depending on the particular condition being treated, various aspects can be selected to achieve the desired results, including number of treatment devices employed, placement of the device(s), frequency of vibrational energy, intensity of vibrational energy, length and frequency of treatment times, and any other characteristics of the vibrational energy applied to the treatment site.

In the case of involuntary tremors, one or more treatment devices may be disposed over the affected limb(s), and relatively weak vibrational energy can be applied. The vibration intensity can be quantified as follows: 25% of the full actuator capacity being weak, 50% being medium, 75% being stronger, 100% being strongest. When using LRA devices, the intensity of the vibration is also affected by the selected frequency. In the example shown in the pictures, 80 Hz is the sweet spot of the actuators providing the maximum intensity when that frequency is selected. In some embodiments, the vibrational energy may include musical patterns (for example, vibrational energy can be mapped from musical input as described in U.S. Patent Publication No. 2017/0098350 A1, which is hereby incorporated by reference in its entirety). Higher frequencies may also be applied. For treatment of dyskinesia, treatment device(s) can be applied over the affected limb, with energy applied at high intensity, and with a frequency of between about 40 Hz and about 120 Hz, for example about 80 Hz.

For freezing of gait, treatment devices can be placed over the user's ankles and continuous vibrational energy can be applied, for example at a frequency of around 100 Hz, with a medium intensity.

For treatment of balance issues, treatment devices can be disposed on the user's wrists and/or ankles and vibrational energy can be applied via a wave pattern (e.g., with intensity increasing and decreasing in a wave-like pattern), with a frequency of, for example, around 50 Hz, with a high intensity.

For treatment of autism, treatment devices can be disposed on a user's wrists, ankles, and/or chest, and the vibrational energy can include a clicking rhythm, which can be synchronized or variable among the devices.

For treatment of PTSD, the treatment devices can be disposed over wrists, ankles, and/or chest, with the vibrational energy having a high intensity with a frequency of around 80 Hz, and a wave-like intensity pattern.

For treatment of high blood pressure, the treatment devices can apply a medium intensity of vibrational energy with variable frequencies, with or without a wave-like intensity pattern.

For treatment of tinnitus, a treatment device can be positioned near the user's ear, and the vibrational energy applied can have a frequency substantially corresponding to the internal frequency heard by the user.

For treatment of spinal cord injuries, one or more treatment devices can be disposed at a suitable location along the back of the neck and/or spine, for example being positioned at or near the location at which the user's sensation ceases (in the case of paralysis or lack of sensation). In some embodiments, the vibrational energy in such places can be applied with variable frequencies.

### Feedback Control Using Sensor Data

As noted previously, in some embodiments delivery of vibrational energy can be modulated based at least in part on feedback obtained via sensor(s). In various examples, this can include sensor(s) carried by the treatment device (e.g., an accelerometer disposed within a housing of the treatment device) or external sensors (e.g., a separately worn fitness tracker, heart monitor device, etc.). Modulation of vibrational energy can include initiating delivery of vibrational energy, ceasing delivery of vibrational energy, or modifying some aspect of the vibrational energy (e.g., frequency, intensity, duty cycle, waveform, etc.).

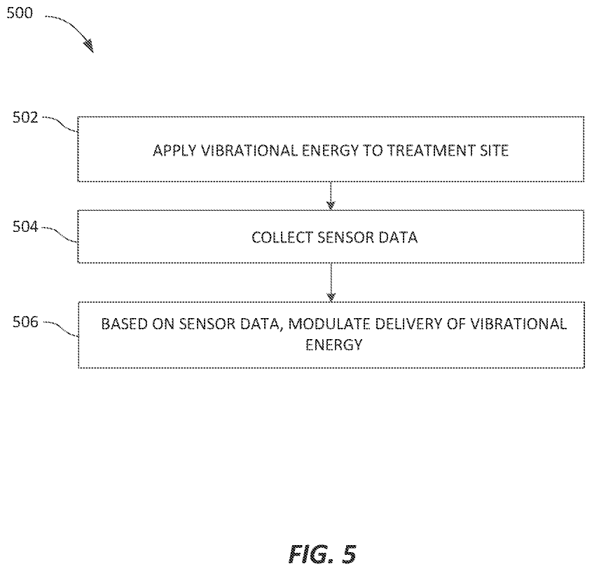

FIG. 5 is a flow diagram of a method for noninvasively modulating nerve activity in accordance with some embodiments. As illustrated, the method 500 includes applying vibrational energy to a treatment site in block 502. For example, a treatment device can be disposed over a user's wrist or other suitable treatment site, and the vibrational energy can be delivered via the vibration actuator(s) disposed within the device.

In block 504, sensor data is collected. As noted previously, this can include using sensors such as accelerometers (or other motion sensors), altimeters, thermometers, blood pressure monitors, cardiac sensors (e.g., ECG sensors), pulse oximeters, blood glucose sensors, or any other suitable sensors that can detect parameters indicative of a user's health or physical condition. In various examples, the sensor data can include a user's movement, body temperature, cardiac rhythm, blood oxygen levels, blood sugar levels, or any other suitable physiological parameter.

In block 506, the delivery of vibrational energy is modulated based at least in part on the sensor data collected in block 504. For example, neuromodulation can be initiated (e.g., the device can begin to apply vibrational energy) in response to certain physiological conditions being met. As another example, the frequency, intensity, duty cycle, or other waveform parameter may be varied in response to detected physiological parameters. In some embodiments, the physiological parameters can be collected across a wide range of users and used to train a machine-learning classification algorithm (e.g., a neural network model) that can be used to determine an appropriate neuromodulation to be applied given a particular parameter or set of parameters detected via the sensor(s). Using Artificial Intelligence (AI) and Machine Learning (ML) algorithms, such behavior can be trained for a more efficient deployment and to avoid false positives.

### Using Machine Learning to Detect Conditions and Manage Treatment

In some embodiments, sensor data can be used to detect physiological parameters or conditions of a user. For example, sensor data can be used to detect the onset of a tremor for a user with Parkinson's. Optionally, such detection can further be used to initiate or modulate delivery of vibrational energy. For example, upon determining that a tremor has begun, the system can identify an appropriate treatment regime and initiate delivery of vibrational energy in accordance with the identified treatment regime. In at least some embodiments, sensor data can continue to be collected during and after treatment, and can be analyzed to determine an efficacy of the treatment, and/or to modulate the ongoing or future delivery of vibrational energy. Although some embodiments described herein relate to treating conditions with vibrational energy, in at least some instances one or more wearable sensors and associated computational resources can be used to detect and/or characterize medical conditions or symptoms without an associated treatment such as vibrational energy. For example, a wearable device with a sensor such as an accelerometer may be configured to detect symptoms or conditions, which may then be used to provide an alert or to pass the information to clinical providers.

As noted previously, in some embodiments, sensor data can be analyzed (e.g., using machine learning or any other suitable technique) to identify symptoms, behaviors, etc. If a symptom is identified (e.g., a Parkinson tremor), an appropriate treatment may be automatically initiated. If treatment is determined to work (e.g., the symptoms disappear or decrease in severity, as determined by sensor data or user input), the treatment may continue. If the treatment is determined not to work (e.g., the symptoms do not decrease in severity or even increase in severity, as determined by sensor data or user input), the treatment regime can be modulated (e.g., applying different frequency, duty cycle, intensity, etc.), and/or a researcher can be alerted.

In various embodiments, any suitable technique may be used for detecting Parkinson tremors, for example using machine learning. Many machine learning techniques are highly computationally expensive, leading to high power consumption. This makes such techniques unsuitable for wearable devices such as the treatment devices described herein. Accordingly, there remains a need for low-power machine learning techniques that can accurately and consistently identify certain conditions or symptoms (e.g., onset of Parkinson tremors) using sensor data from wearable devices.

In some embodiments, the treatment system described above (e.g., including one or more individual treatment devices, each having one or more vibration actuators, one or more sensors, and a controller) can be used to accurate classify tremor conditions using algorithms developed by machine learning techniques. For example, a classifier model can be developed that is specific to a particular patient (or some patient population) and/or that is particular to a particular treatment device location. For example, a right wrist treatment device may utilize a first algorithm to detect tremor conditions, while a left wrist treatment device may utilize a second, different algorithm to detect tremor conditions. This approach can be useful where the signature patient movements (and therefore the corresponding sensor data) is different between different parts of a user's body (e.g., wrists vs. hands, left vs. right side, etc.).

In some examples, for a particular user and a particular treatment device, sensor data can be collected under controlled conditions, for example in a clinician's office when a user is known to be in a non-tremor condition (i.e., not experiencing any tremors or related symptoms) and when a user is known to be in a tremor condition (i.e., experiencing any tremors or related symptoms). In some examples, a clinician may use a paired application to designate certain time periods as corresponding to tremor conditions, and the sensor data collected during those time periods can be tagged accordingly. A resulting data set including both non-tremor data and tremor data can then be used to train a classifier model using machine learning techniques. In some embodiments, sensor data can be collected via the treatment devices and transmitted to one or more remote computing devices that are more computationally capable, and therefore more well suited to executing the machine learning operation. The output of the machine learning operation, however, may be a classifier (e.g., an algorithm that can be used to identify tremor conditions based on the sensor data in real-time or nearly real-time) that is compact and computationally efficient enough to be executed locally by the treatment devices themselves. Additionally or alternatively, the classifier can be executed via a local device that is communicatively coupled with the treatment device(s), such as a smartphone running an application that is wirelessly coupled to the treatment devices. In operation, the treatment devices can transmit sensor data to the smartphone or other local device, which may then utilize the classifier to determine whether a tremor condition is satisfied. If the classifier determines that a tremor condition is satisfied, then the treatment device may initiate delivery of vibrational energy.

In some instances, the system may initiate delivery of vibrational energy even when the user is not actually experiencing a tremor. In such “false positive” conditions, in which the classifier has erroneously detected a tremor condition even though the user is not in fact experiencing a tremor, it can be useful to allow for a user input to disable the delivery of vibrational energy. In some embodiments, for example, a user may utilize a user interface component of the treatment device or an associated local device to terminate delivery of vibrational energy. This can include, for example, providing a haptic input (e.g., double tapping on the treatment device), providing a user input via an associated application running on a user's smartphone or other local device, speaking a certain phrase or keyword, or any other suitable user input. In some embodiments, such false-positive designations can be stored and later used to update the classifier model (e.g., the sensor data collected during the time preceding the user intervention to terminate treatment can be flagged as non-tremor data, and the machine learning process may be repeated and/or updated to generate a revised classifier model).

By training individualized models for tremor identification, both at the user level and at the device location level, the efficacy of the detection increases compared to a generalized model, hence reducing false positives and negatives. Additionally, using both accelerometer and gyroscope data can further increase the precision of the tremor condition determinations.

While there have been attempts to detect tremors using machine learning techniques, none have provided a combination of algorithm, data conditioning, separation of training/inference, and sufficiently simplified pruning of the model to obtain a result of inference while using low power (e.g., less than about 1 milliwatt, less than about 1 microwatt, etc.). For the data collection, it can be beneficial to collect, condition and label the sensor data (e.g., inertia signal) in a manner that provides a high confidence model after training to detect a tremor, and without penalizing power consumption. This includes the correct selection of the output data rate to be high enough to detect the gesture of a tremor, yet not so high as to fail in the power consumption. This further includes the selection of the correct energy collection for the accelerometer and gyroscope, in the right coordinates, and the correct selection of signal filters for the accelerometer and gyroscope data for signal conditioning. For the training, it is beneficial to select a sufficiently simplified classifier algorithm, that will create a model that can be implemented in a very resource-constrained treatment device.

In some embodiments, the classifier algorithm can take the form of a decision tree, in which features extracted from sensor data (e.g., acceleration along particular axes, rotation along particular axes, and derivatives thereof, etc.) are provided as inputs and the output is binary determination of a tremor condition and non-tremor condition. Decision tree algorithms come in many forms, such as C4.5 and C5.0 implementations, although other variations can also comply with the low power inference goal. As a result of the training, a very efficient and low-power model is generated that can be executed for inference at very low power.

In some embodiments, the sensor of the treatment device can include an inertia motion unit (IMU) configured to collect accelerometer and gyroscope data, and which may also perform some functions of a controller. One example of such an IMU is ST Microelectronics' LSM6DSOX, which may have suitable capacity to condition the signals generated, to use filters and internal calculation of its own IMU data, and to compare the conditioned data against the trained model and infer when the tremor happens, generating an interrupt that will signal the vibration actuator of the treatment device.

In some embodiments, the software tool to interface with and program the IMU may be the Uncleo-GUI provided by ST Microelectronics. Using this tool, filters for signal conditioning can be designed, attributes of the data can be calculated (e.g., as the root mean square (RMS) energy of a certain axis, like gyroscope X, or any other parameter that is at least partially based upon motion data), as well as other parameters necessary to obtain the appropriate samples, like the ODR (frequency of the samples) and the sampling time window. Each one of the different conditionings may be referred to as a feature.

After the selection for the data conditioning, the sensor data is effectively collected from the treatment device. The feature extraction (e.g., calculating the RMS energy of a certain axis) can be performed by the treatment device, or alternatively may be performed by a locally paired device such as a smartphone.

Below is an abbreviated example of sensor data captured during training of a classifier model. The following include two sets of labeled data: a first set labeled as “non-tremor” and a second set labeled as “tremor.” For each, the sensor data includes the acceleration along each of three axes and the rotation about each of three axes.

Non-Tremor Data

| time [ms] | accX[mg] | accY[mg] | accZ [mg] | gyroX[mdps] | gyroY[mdps] | gyroZ[mdps] |
|---|---|---|---|---|---|---|
| 36415930 | 45 | 239 | 977 | 6842 | 1373 | −2318 |
| 36415970 | 52 | 235 | 967 | 4182 | 1120 | −1505 |
| 36416010 | 41 | 227 | 976 | 3850 | 1120 | −1785 |
| 36416050 | 46 | 234 | 967 | 4970 | −245 | −2415 |

Tremor Data

| time [ms] | accX[mg] | accY[mg] | accZ[mg] | gyroX[mdps] | gyroY[mdps] | gyroZ[mdps] |
|---|---|---|---|---|---|---|
| 36526820 | 595 | 571 | 464 | −39147 | 12416 | 4357 |
| 36526860 | 584 | 504 | 456 | −5022 | 1417 | 472 |
| 36526900 | 625 | 575 | 406 | 37633 | −10062 | −5355 |
| 36526940 | 625 | 575 | 406 | 44117 | −11322 | −8995 |

This time-series data can then be used to generate a feature document that can later be used for classification and training. As noted above, such training can be performed remotely (i.e., on hardware other than the treatment devices themselves) in order to take advantage of greater computational capacity of those devices. The feature document can include features that are based on the sensor data noted above. Examples include, for example, absolute mean, mean, variance, energy calculations, peak-to-peak values, or any other values that can be extracted from the raw time-series sensor data. In various embodiments, each feature can be a filter and or calculation of a time window of the sensor data. In operation, each of these features can output a particular value at a particular time, and the time-series data that results can be used by a classifier such as a decision tree algorithm to discriminate between tremor conditions and non-tremor conditions.

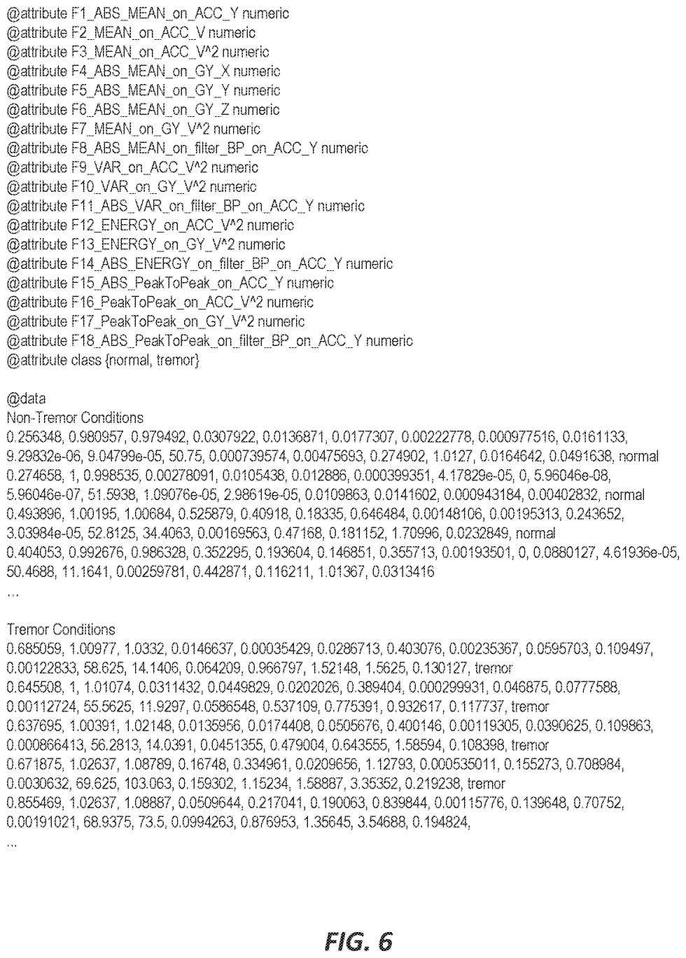

FIG. 6 illustrates example features (labeled as “attributes”) that can be extracted from the sensor data, along with example data for these features under non-tremor and tremor conditions. As an example, the feature “@attribute F18_ABS_PeakToPeak_on_filter_BP_on_ACC_Y numeric” indicates that on the Y axis of the acceleration, a bandpass filter is applied, and after that a formula calculation of the absolute of the peak-to-peak value is used as a result.

In various embodiments, any suitable algorithm can be used for developing a classifier. Among examples, a decision tree model can be generated and trained based on the collected sensor data. Decision tree methods can construct a model of decisions made based on actual values of attributes in the data. In this arrangement, decisions fork in tree structures until a prediction decision is made for a given record. Decision trees are trained on data for classification and regression problems. Decision trees can be both fast and accurate. Importantly, even if training of the decision tree is computationally intensive, the inferences generated by use of the resulting decision tree can be calculated very power-efficiently.

Popular decision tree algorithms include classification and regression tree (CART), iterative dichotomiser 3 (ID3), C4.5 and C5.0, chi-squared automatic interaction detection (CHAID), decision stump, M5, and conditional decision trees. In various embodiments, any suitable decision tree algorithm, or other suitable classifier algorithm, can be used.

In one example, a suitable software tool for data classification is Waikato Environment for Knowledge Analysis (WEKA), which is an open-source machine learning tool that can implement, among other things, the C4.5 algorithm, which is a classification algorithm that produces decision trees based on information theory. The decision trees generated by C4.5 are used for classification, and for this reason, C4.5 is often referred to as a statistical classifier. In some implementations, the C4.5 algorithm may also have additional features such as accounting for missing values, decision trees pruning, continuous attribute value ranges, derivation of rules, etc. In the WEKA data mining tool, J48 is an open-source Java implementation of the C4.5 algorithm. J48 allows classification via either decision trees or rules generated from them.

As an output of the classification process, the result includes the confusion matrix, the accuracy by class, and the decision tree that will can be used to infer classification of future data. This decision tree, which can be relatively compact and computationally efficient to execute, may then be run locally by each treatment device based on the real-time sensor data captured by that particular device. In this way, the machine-learned decision tree can be used to make real-time determinations of tremor conditions, and to initiate or modulate delivery of vibrational energy in response. The following is an example decision tree generated by the sample data as shown in FIG. 6:

F9_VAR_on_ACC_V^2<=0.035156: non-tremor (33.0)

F9_VAR_on_ACC_V^2>0.035156

| F10_VAR_on_GY_V^2<=6.89844

| | F1_ABS_MEAN_on_ACC_Y<=0.305664: non-tremor (5.0)

| | F_1_ABS_MEAN_on_ACC_Y>0.305664

| | | F1_ABS_MEAN_on_ACC_Y<=0.875977

| | | | F9_VAR_on_ACC_V^2<=0.041016

| | | | | F4_ABS_MEAN_on_GY_X<=0.186401: tremor (3.0)

| | | | | F4_ABS_MEAN_on_GY_X>0.186401: non-tremor (2.0)

| | | | F9_VAR_on_ACC_V^2>0.041016: tremor (33.0)

| | | F1_ABS_MEAN_on_ACC_Y>0.875977: non-tremor (3.0)

| F10_VAR_on_GY_V^2>6.89844: non-tremor (8.0)

This tree has seven “leaves,” which are endpoints which indicate either a tremor or non-tremor condition. In operation, a tree such as that shown above can be downloaded to an individual treatment device (e.g. a left-wrist device for a particular user), which may then continuously calculate the decision tree based on sensor data collected by the treatment device to infer whether the detected movement belongs to the set of tremor conditions or non-tremor conditions.

### IV. Conclusion

Although many of the embodiments are described above with respect to systems, devices, and methods for noninvasive neuromodulation, the technology is applicable to other applications and/or other approaches, such as noninvasive application of vibrational energy for other non-neuromodulation applications, such as to augment musical listening experiences, as interactive multi-media, or any other application. Additionally, systems, devices, and methods of the present technology can be applied for therapeutic effect that does not involve (or does not primarily rely on) neuromodulation. For example, in some embodiments the vibrational actuators may instantiate one or more therapeutic effects independent of modulating nerve activity. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-6.

The above detailed descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Where a range of values is provided, it is understood that each intervening value, to the tenth of the unit of the lower limit unless the context clearly dictates otherwise, to between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A noninvasive treatment system, comprising a plurality of treatment devices each configured to be disposed over a respective treatment site of a user, wherein each of the treatment devices comprises:
a vibration actuator configured to deliver vibrational energy to the respective treatment site of the user;
one or more sensors configured to obtain physiological data from the user, the physiological data including at least movement data; and
a controller communicatively coupled to the one or more sensors, the controller configured to:
receive the physiological data from the one or more sensors;
analyze the physiological data to determine that a tremor condition has been detected;
based on the determination that the tremor is occurring, initiate delivery of vibrational energy via the vibration actuator;
while delivering vibrational energy via the vibration actuator, receive additional physiological data from the sensor;
analyze the additional physiological data to determine that the tremor has ceased or decreased in severity; and
based on the determination that the tremor condition is no longer detected, ceasing the delivery of vibrational energy via the vibration actuator.

2. The noninvasive treatment system of claim 1, wherein analyzing the physiological data to determine that a tremor is occurring comprises using a decision tree to evaluate the physiological data.

3. The noninvasive treatment system of claim 1, wherein each of the treatment devices further comprises an input mechanism, and wherein the controller is further configured to:
while delivering vibrational energy via the vibration actuator, receiving a user input via the input mechanism; and
responsive to the user input, ceasing the delivery of vibrational energy via the vibration actuator.

4. The noninvasive treatment system of claim 3, wherein the input mechanism comprises a touch-sensitive element, and wherein the user input comprises a user tapping the touch-sensitive element.

5. The noninvasive treatment system of claim 1, wherein the controller is an ultra-low power controller configured to analyze the physiological data to determine that the tremor is occurring using less than about 1 milliwatt of power.

6. The noninvasive treatment system of claim 5, wherein the controller is an ultra-low power controller configured to analyze the physiological data to determine that the tremor is occurring using less than about 1 microwatt of power.

7. The noninvasive treatment system of claim 1, wherein the one or more sensors comprises an accelerometer and a gyroscope, and wherein the physiological data comprises accelerometer motion data along three axes and gyroscope rotation data along three axes.

8. The noninvasive treatment system of claim 1, wherein at least some of the treatment devices comprise a housing coupled to a fastener configured to secure the housing against the user's wrists or ankles.

9. The noninvasive treatment system of claim 5, wherein the housing encloses the vibration actuator, the one or more sensors, and the controller.

10. The noninvasive treatment system of claim 1, wherein the treatment devices comprises at least four treatment devices configured to be disposed over a user's wrists and ankles, respectively, and wherein each of the treatment devices collects and analyzes physiological data independently of the other treatment devices.

11. A treatment device, comprising:
a vibration actuator configured to deliver vibrational energy to a treatment site of a user;
one or more sensors configured to obtain physiological data from the user; and
a controller communicatively coupled to the one or more sensors, the controller configured to:
receive the physiological data from the one or more sensors;
analyze the physiological data; and
based on the analysis, modulate delivery of vibrational energy via the vibration actuator.

12. The treatment device of claim 11, wherein analyzing the physiological data comprises applying a classification algorithm to the physiological data to make a tremor determination.

13. The treatment device of claim 11, wherein modulating delivery comprises initiating delivery of vibrational energy via the vibration actuator.

14. The treatment device of claim 11, wherein modulating delivery comprises at least one of: initiating delivery of vibrational energy via the vibration actuator, ceasing delivery of vibrational energy via the vibration actuator, varying an intensity of vibrational energy delivered via the vibration actuator, or varying a frequency of vibrational energy delivered via the vibration actuator.

15. The device of claim 11, wherein the one or more sensors comprises at least one of: an accelerometer, a gyroscope, a temperature sensor, or a blood pressure sensor.

16. The device of claim 11, wherein the physiological data comprises an indication of a user tremor.

17. A method for treatment of a tremor, the method comprising:
disposing a wearable treatment device adjacent a treatment site of the user, the treatment device comprising a vibration actuator, one or more sensors, and a controller;
sensing physiological data via the one or more sensors of the treatment device, the physiological data including at least movement data;
analyzing, via the controller, the physiological data to make a determination that a tremor condition has been detected;
after making the determination, applying vibrational energy to the treatment site via the vibration actuator of the treatment device;
sensing additional physiological data of the user via the one or more sensors of the treatment device;
analyzing, via the controller, the additional physiological data to make a determination that the tremor condition is no longer detected; and
after making the determination that the tremor condition is no longer detected, ceasing applying vibrational energy to the treatment site via the vibration actuator of the treatment device.

18. The method of claim 17, wherein the treatment device further comprises an input mechanism, and wherein the method further comprises:
while applying vibrational energy via the vibration actuator, receive a user input via the input mechanism; and
responsive to the user input, ceasing applying vibrational energy via the vibration actuator.

19. The method of claim 17, wherein the controller consumes less than about 1 milliwatt of power in analyzing the physiological data.

20. The method of claim 17, wherein the one or more sensors comprises an accelerometer and a gyroscope, wherein the physiological data comprises accelerometer motion data along three axes and gyroscope rotation data along three axes, and wherein analyzing the physiological data comprises applying a decision tree to the accelerometer motion data and the gyroscope rotation data.
